SEQUENCE LISTING (6 sequences)

SEQ ID NO: 1 - DNA, 333 bp, Artificial Sequence
  Synthetic inducible promoter
tcgaatgagtcacatcgatctccgccccctcttcgagggggcggggtcgaggaggaaaaactcgaatgagtcacatcgaccctttgatcttcgaggggactttccggggtggagcaagcgtgacaagtccacgtatgacccgaccgacgatatcgaagcctacgcgctgaacgccagccccgatcgaccccgccccctcgatttccaagaaatcgaatgacatcatctttcgaatgacatcatctttcgaggggactttcctcgaacttccttcgaggggactttcctcgaggggactttcctcgaggaggaaaaactcgagtagagtctaga

SEQ ID NO: 2 - DNA, 390 bp, Artificial Sequence
  Synthetic inducible promoter
ctgcttagggttaggcgttttgcgctgcttcgcgatcgaatgagtcacatcgatctccgccccctcttcgagggggcggggtcgaggaggaaaaactcgaatgagtcacatcgaccctttgatcttcgaggggactttccggggtggagcaagcgtgacaagtccacgtatgacccgaccgacgatatcgaagcctacgcgctgaacgccagccccgatcgaccccgccccctcgatttccaagaaatcgaatgacatcatctttcgaatgacatcatctttcgaggggactttcctcgaacttccttcgaggggactttcctcgaggggactttcctcgaggaggaaaaactcgagtagagtctagactctacattttgacacccccat

SEQ ID NO: 3 - DNA, 283 bp, Artificial Sequence
  Synthetic inducible promoter
ctgcttagggttaggcgttttgcgctgcttcgcgatcgaagatcaaagggtcgatttcttggaaatcgatgtgactcattcgatcacgtcctcgaggaggaaaaactcgaggaaagtcccctcgaacttccttcgagggggcggggtcgaatgagtcacatcgaggaaagtcccctcgaggggactttcctcgatttcttggaaatcgaagagggggcggagatcgagtttttcctcctcgaggaaagtcccctcgatcgactctacattttgacacccccat

SEQ ID NO: 4 - DNA, 252 bp, Artificial Sequence
  Synthetic inducible promoter
ctgcttagggttaggcgttttgcgctgcttcgcgatcgaggaaagtcccctcgaggaaagtcccctcgatttcttggaaatcgaatgacatcatctttcgatcacgtcctcgaggaaagtcccctcgagtttttcctcctcgaggggactttcctcgatttccaagaaatcgatttcttggaaatcgaccctttgatcttcgaggaggaaaaactcgagtagagtctagactctacattttgacacccccat

SEQ ID NO: 5 - DNA, 272 bp, Artificial Sequence
  Synthetic inducible promoter
ctgcttagggttaggcgttttgcgctgcttcgcgatcgaggaggaaaaactcgatttcttggaaatcgaggggactttcctcgaaagatgatgtcattcgaagatcaaagggtcgatgtgactcattcgaggggactttcctcgagggggcggggtcgaatgacatcatctttcgaggggactttcctcgaggggactttcctcgaatgacatcatctttcgatttcttggaaatcgagtagagtctagactctacattttgacacccccat

SEQ ID NO: 6 - DNA, 29 bp, Artificial Sequence
  HS-A insulator
tgagatctttcacaaaacaccagttatgc

(19) United States
(12) Patent Application Publication
(10) Pub. No.: US 2023/0242936 A1
(43) Pub. Date: Aug. 3, 2023

(54) DUAL PROMOTER SYSTEMS

(71) Applicant: Seattle Children's Hospital (dba Seattle Children's Research Institute), Seattle, WA (US)

(72) Inventors: Michael C. Jensen, Seattle, WA (US); Jia Wei, Seattle, WA (US)

(21) Appl. No.: 17/924,811

(22) PCT Filed: May 12, 2021
(86) PCT No.: PCT/US2021/031986

§ 371 Date: Nov. 11, 2022

Related U.S. Application Data
(60) Provisional application No. 63/024,946, filed on May 14, 2020.

(51) Int. Cl. C12N 15/85; C12N 15/63; C12N 5/0783; C12N 9/22; C12N 15/90; A61K 48/00

(52) U.S. Cl. CPC C12N 15/85; C12N 15/635; C12N 5/0636; C12N 9/22; C12N 15/907; A61K 48/0066; C12N 2830/002; C12N 2830/20; C12N 2800/90

(57) ABSTRACT

Some embodiments of the methods and compositions provided herein relate to systems comprising a promoter operably linked to a nucleic acid encoding a receptor, and an inducible promoter operably linked to a nucleic acid encoding a payload. In some embodiments, transcription from the inducible promoter is induced by activation of the receptor. In some embodiments, transcription from the inducible promoter is further modulated by inhibiting a signal between the activated receptor and the inducible promoter.

### CROSS-REFERENCE TO RELATED APPLICATIONS

This application claims priority to U.S. Prov. App. No. 63/024946 filed May 14, 2020 entitled “DUAL PROMOTER SYSTEMS,” which is hereby expressly incorporated by reference in its entirety.

### REFERENCE TO SEQUENCE LISTING

The present application is being filed along with a Sequence Listing in electronic format. The Sequence Listing is provided as a file entitled SCRI247WOSEQLIST, created May 6, 2021, which is approximately 3 Kb in size. The information in the electronic format of the Sequence Listing is incorporated herein by reference in its entirety.

### FIELD OF THE INVENTION

Some embodiments of the methods and compositions provided herein relate to systems comprising a nucleic acid including a promoter operably linked to a nucleic acid encoding a receptor, and an inducible promoter operably linked to a nucleic acid encoding a payload. In some embodiments, transcription from the inducible promoter is induced by activation of the receptor. In some embodiments, transcription from the inducible promoter is further modulated by inhibiting a signal between the activated receptor and the inducible promoter.

### BACKGROUND

Gene expression from chromosomal expression constructs that have two or more gene expression cassettes is confounded by interference from the surrounding chromosomal environment as well as “cross-talk” between the transcription factors at the gene expression cassettes. Accordingly, the need for additional constitutive or inducible gene expression systems is manifest.

### SUMMARY

Some embodiments of the methods and compositions provided herein include a nucleic acid encoding a dual promoter system, comprising: a first polynucleotide comprising an inducible first promoter operably linked to a polynucleotide encoding a payload, wherein transcription from the first promoter is inducible by activation of a receptor; a second polynucleotide comprising a second promoter operably linked to a polynucleotide encoding the receptor; and wherein a first insulator is located between the first polynucleotide and the second polynucleotide.

In some embodiments, the first promoter comprises a nucleotide sequence having at least 95% identity with the nucleotide sequence set forth in any one of SEQ ID NOs:1-5. In some embodiments, the first promoter comprises the nucleotide sequence set forth in SEQ NO:1.

In some embodiments, the first promoter further comprises an IL-2 minimal promoter.

In some embodiments, the second promoter is constitutive. In some embodiments, the second promoter is selected from the group consisting of an EF1α promoter, a PGK promoter, a MND promoter, a MSCV promoter, a CMV promoter, a UBC promoter, a CAGE promoter, and an SV40 promoter. In some embodiments, the second promoter comprises an EF1α promoter.

In some embodiments, the first insulator comprises an enhancer-blocking insulator. In some embodiments, the first insulator is selected from the group consisting of a 3′ HS-A β-globin insulator, an 5′ HS4β-globin insulator, an 5′ HS5 insulator, an scs insulator, an scs' insulator, a gypsy insulator, a CTCF insulator, a scaffold attachment region (SAR) insulator, an DMD/ICR insulator, an apoB insulator, a DM1 insulator, a BEAD-1 insulator, a HS2-6 insulator, a DMD/ICR insulator, a Lys 5′ A insulator, an RO insulator, a UR1 insulator, an sns insulator, a Fab-7 insulator, a Fab-8 insulator, a faswb insulator, an eve promoter insulator, an HMR tRNAThr insulator, a Chal UAS insulator, an UASrpg insulator, and a STAR insulator. In some embodiments, the first insulator comprises a 3′ HS-A β-globin insulator. In some embodiments, the first insulator comprises a nucleotide sequence having at least 95% identity with the nucleotide sequence set forth in SEQ ID NO:6.

In some embodiments, the receptor is activated by the receptor binding a ligand for the receptor.

In some embodiments, the receptor comprises a cell surface receptor. In some embodiments, the receptor comprises a cell receptor (TCR). In some embodiments, the receptor comprises a chimeric antigen receptor (CAR). In some embodiments, the CAR comprises an intracellular signaling domain comprising a costimulatory domain selected from the group consisting of CD27, CD28, 4-IBB, OX-40, CD3O, CD40, PD-1, ICOS, LFA-1, CD2, CD7, NKG2C, and B7-H3, preferably in combination with a CD3-zeta domain or a functional portion thereof. in some embodiments, the intracellular signaling domain comprises a 4-1BB costimulatory domain. In some embodiments, the intracellular signaling domain comprises a CD28 cytoplasmic domain.

In some embodiments, the second polynucleotide comprises a polynucleotide encoding a selectable marker. In some embodiments, the selectable marker comprises a cell-surface selectable marker. In some embodiments, the cell-surface selectable marker is selected from a truncated EGFR (EGFRt) polypeptide and a truncated Her2 (Her2t) polypeptide. In some embodiments, the selectable marker comprises a drug selectable marker. In some embodiments, the drug selectable marker comprises a DHFR polypeptide.

In some embodiments, a ribosome skip sequence or an internal ribosome entry site (TRES) is located between the polynucleotide encoding the receptor and the polynucleotide encoding a selectable marker. In some embodiments, the ribosome skip sequence is selected from the group consisting of a P2A sequence, a T2A sequence, an E2A sequence, and an F2A. sequence.

In some embodiments, the first polynucleotide and the second polynucleotide are located between a second insulator and a third insulator. In some embodiments, the second insulator and/or third insulator comprises an enhancer-blocking insulator. In some embodiments, the second insulator and/or third insulator is selected from the group consisting of a 3′ HS-A β-globin insulator, an 5′ HS4 β-globin insulator, an 5′ HS5 insulator, an scs insulator, an scs' insulator, a gypsy insulator, a CTCF insulator, a scaffold attachment region (SAR) insulator, an DMD/ICR insulator, an apoB insulator, a DM1 insulator, a BEAD-1 insulator, a HS2-6 insulator, a DMD/ICR insulator, a Lys 5′ A insulator, an RO insulator, a UR1 insulator, an sns insulator, a Fab-7 insulator, a Fab-8 insulator, a faswb insulator, an eve promoter insulator, an MIR tRNAThr insulator, a Chat UAS insulator, an UASrpg insulator, and a STAR insulator. In some embodiments, the second insulator and/or third insulator comprises a 5′ HS4 β-globin insulator.

In some embodiments, the first polynucleotide and/or second polynucleotide comprises a poly A tail.

In some embodiments, the payload comprises a first protein or a first non-coding RNA. In some embodiments, the payload comprises a second protein or a second non-coding RNA. In some embodiments, a ribosome skip sequence or an internal ribosome entry site (IRES) is located between a polynucleotide encoding the first protein or the first non-coding RNA and a polynucleotide encoding the second protein or the second non-coding RNA.

In some embodiments, the payload comprises an intracellular protein, a transmembrane protein, a membrane bound protein, a secreted protein, or a microRNA (miRNA). In some embodiments, the payload comprises a signal transducer and activator of transcription (STAT) protein, or a functional derivative of a STAT protein. In some embodiments, the STAT protein comprises a constitutively activated STAT (CA-STAT) protein. In some embodiments, the STAT protein comprises a STAT5a protein. In some embodiments, the STAT protein comprises a fusion protein comprising a functional portion of a constitutively activated STAT5a and of an estrogen receptor.

Some embodiments of the methods and compositions provided herein include a pharmaceutical composition comprising any one of the nucleic acids provided herein and a pharmaceutically acceptable excipient.

Some embodiments of the methods and compositions provided herein include a vector comprising any one of the nucleic acids provided herein. In some embodiments, the vector comprises a transposon vector, a viral vector, a plasmid, a nanoplasmid, or a minicircle. In some embodiments, the vector comprises a lentiviral vector. In some embodiments, the vector comprises a transposon sequence. In some embodiments, the transposon sequence comprises a PiggyBac transposon sequence, or a Sleeping Beauty transposon sequence. Some embodiments also include a polynucleotide sequence encoding a self-inactivating transposase.

Some embodiments of the methods and compositions provided herein include a host cell comprising any one of the nucleic acids provided herein.

In some embodiments, the host cell is an immune cell.

In some embodiments, the host cell is a precursor T cell, or a hematopoietic stern cell.

In some embodiments, the host cell is a T cell, a B cell, a natural killer cell, an antigen presenting cell, a dendritic cell, a macrophage, or a granulocyte such as a basophil, an eosinophil, a neutrophil, or a mast cell.

In some embodiments, the host cell is a CD4+ cell or a CD8+ T cell. In some embodiments, the host cell is a CD8+ cytotoxic cell selected from the group consisting of a naïve CD8+ cell, a CD8+ memory T cell, a central memory CD8+ T cell, a regulatory CD8+ T cell, an iPS derived CD8+ cell, an effector memory CD8+ T cell, and a bulk CD8+ T cell. In some embodiments, the host cell is a CD4+ helper cell selected from the group consisting of a naïve CD4+ T cell, a CD4+ memory T cell, a central memory CD4+ cell, a regulatory CD4+ T cell, an iPS derived CD4+ T cell, an effector memory CD4+ T cell, and a bulk CD4+ T cell.

In some embodiments, the host cell is allogenic to a subject, or is autologous to a subject. In some embodiments, the host cell is ex vivo. In some embodiments, the host cell is in vivo.

In some embodiments, the host cell is mammalian. In some embodiments, the host cell is human.

Some embodiments of the methods and compositions provided herein include a pharmaceutical composition comprising any one of the host cells provided herein and a pharmaceutically acceptable excipient.

Some embodiments of the methods and compositions provided herein include a method of treating, ameliorating, or inhibiting, a disorder in a subject, comprising administering any one of the host cells provided herein to the subject in need thereof.

Some embodiments also include reducing transcription from the first promoter by administering an inhibitor to the subject, wherein an intracellular signal between the activated receptor and the first promoter induces transcription from the first promoter, and wherein the inhibitor blocks the intracellular signal. In some embodiments, the inhibitor comprises a tyrosine kinase inhibitor. In some embodiments, the inhibitor comprises dasatinib.

Some embodiments also include increasing transcription from the first promoter by administering an activator to the subject, wherein the activator induces transcription from the first promoter. In some embodiments, the activator comprises a tyrosine kinase activator. In some embodiments, the activator is selected from phorbol 12-myristate 13-acetate (PMA) or lonomycin.

In some embodiments, the disorder is selected from the group consisting of a cancer, an inflammatory disorder, a viral infection, a fungal infection, and a bacterial infection.

In some embodiments, the receptor comprises a chimeric antigen receptor (CAR). In some embodiments, the CAR specifically binds to an antigen selected from the group consisting of an antigen expressed on the surface of a cancer cell, a viral antigen, a fungal antigen, or a bacterial antigen. In some embodiments, the CAR specifically binds to an antigen selected from CD19 and. CD171.

In some embodiments, the disorder comprises a cancer comprising a leukemia, a breast cancer, a stomach cancer, an esophageal cancer, a brain cancer, a uterine cancer, a prostate cancer, a bone cancer, a liver cancer, a pancreatic cancer, an ovarian cancer, a lung cancer, a colon cancer, a kidney cancer, a bladder cancer, or a thyroid cancer.

In some embodiments, the subject is mammalian. In some embodiments, the subject is human.

Some embodiments of the methods and compositions provided herein include a method for modulating activity of a host cell, comprising: providing any one of the host cells provided herein, wherein the receptor comprises a chimeric antigen receptor (CAR); and activating the CAR by contacting the CAR with a ligand, wherein the CAR specifically binds to the ligand, thereby inducing transcription from the first promoter.

Some embodiments also include reducing transcription from the first promoter by contacting the host cell with an inhibitor, wherein an intracellular signal between the activated CAR and the first promoter induces transcription from the first promoter, and wherein the inhibitor blocks the intracellular signal. In some embodiments, the inhibitor comprises a tyrosine kinase inhibitor. In some embodiments, the inhibitor comprises dasatinib.

Some embodiments also include increasing transcription from the first promoter by contacting the host cell with an activator, wherein the activator induces transcription from the first promoter. In some embodiments, the activator comprises a tyrosine kinase activator. In some embodiments, the activator is selected from phorbol 12-myristate 13-acetate (PMA) or ionomycin.

Some embodiments also include inducing or increasing an activity of the payload by contacting the host cell with a payload activator. In some embodiments, the payload comprises a fusion protein comprising a functional portion of a constitutively activated signal transducer and activator of transcription 5a (CA-STAT5a) protein and of an estrogen receptor. In some embodiments, the payload activator comprises tamoxifen, an analogue of tamoxifen, a metabolite of tamoxifen, or derivative thereof.

Some embodiments of the methods and compositions provided herein include a system comprising a host cell comprising: the nucleic acid of any one of the nucleic acids provided herein; and the receptor. In some embodiments, the receptor is activated.

Some embodiments also include an inhibitor, wherein an intracellular signal between the activated receptor and the first promoter induces transcription from the first promoter, and wherein the inhibitor blocks the intracellular signal. In some embodiments, the inhibitor comprises a tyrosine kinase inhibitor. In some embodiments, the inhibitor comprises dasatinib.

Some embodiments also include a payload activator, wherein the payload activator induces or increases an activity of the payload. In some embodiments, the payload comprises a fusion protein comprising a functional portion of a constitutively activated signal transducer and activator of transcription 5a (CA-STAT5a) protein and of an estrogen receptor.

In some embodiments, the payload activator comprises tamoxifen, an analogue of tamoxifen, a metabolite of tamoxifen, or derivative thereof.

In some embodiments, the receptor comprises a chimeric antigen receptor (CAR).

In some embodiments, the host cell is an immune cell. In some embodiments, the host cell is allogenic to a subject, or is autologous to a subject. In some embodiments, the host cell is ex vivo. In some embodiments, the host cell is in vivo.

In some embodiments, the host cell is mammalian. In some embodiments, the host cell is human.

### BRIEF DESCRIPTION OF THE DRAWINGS

FIG. 1 depicts a schematic of a dual promoter construct and includes a 5′ HSA insulator; an inducible synthetic promoter (iSynPro); an IL-2 minimal promoter (IL-2mp); a polynucleotide encoding a payload green fluorescent protein/luciferase protein (GFP:ffluc); an RBG-poly A tail; a 3′ HS-A insulator; a constitutive EF1α promoter (EF1a); a polynucleotide encoding an anti-CD19 chimeric antigen receptor (CD19CAR); a P2A ribosome skip sequence; a polynucleotide encoding a dihydrofolate reductase protein with two mutations (DHFRdm) to increase its efficiency as a selectable marker; a T2A ribosome skip sequence; a polynucleotide encoding a truncated EGFR (EGFRt) polypeptide; a BGH poly A tail; a 5′ HSA insulator; a PiggyBac 3′ terminal repeat sequence; a trpA; an R6K mini origin; an RNA-OUT; and a PiggyBac 5′ terminal repeat sequence.

FIG. 2 depicts a FACS analysis for expression of EGFRt from a constitutive promoter and expression of GFP from an inducible iSynPro promoter in primary CD8+ anti-CD19 CAR T cells prepared by either electroporation with a single dual promoter construct (left panels), or by co-transduction with lentiviral constructs (right panels). Transfected cells were co-cultured in the presence of cells expressing tumor antigen (CD19) to activate the anti-CD19 CARs.

FIG. 3 depicts a FACS analysis for expression of 4-1BB or GFP in anti-CD19 CAR T cells prepared by co-transduction with lentiviral constructs containing a constitutively expressed anti-CD19 CAR, and an inducibly expressed GFP. The cells were treated under various conditions in the absence or presence of 100 nM dasatinib, including: (1) cultured in the presence phorbol 12-myristate 13-acetate (PMA) and lonomycin, which induced transcription from the inducible iSynPro promoter; (2) cultured in the presence of antiCD3/CD28, which activates the T-cells; (3) co-cultured with K562 cells, which do not express CD19; (4) co-cultured with CD19⁻ K562/OKT3 cells, which activate the TCR; (5) co-cultured with CD19+ K562 cells, which activate the CD19 CAR; and (6) co-cultured with CD19+ Raji cells, which activate the CD19 CAR.

FIG. 4 depicts an experimental protocol for a xenograft model.

FIG. 5 depicts images of treated mice from day 1 to day 9 for bioluminescent signal.

FIG. 6 depicts a line graph of bioluminescent signal over time for treated mice, with average line graphs in bold.

FIG. 7A depicts a dual promoter construct containing an inducible iSynPro promoter and IL2 minimal promoter operably linked to a nucleic acid encoding caSTAT5a, a T2A sequence, and a truncated HER2 (Her2t) marker protein; an insulator sequence; a constitutive EF1a promoter operably linked to a nucleic acid encoding an anti-CD19 CAR, a P2A sequence, a selectable DHFRdin protein, a T2A sequence, and a truncated EGFR (EGFRt) marker protein.

FIG. 7B depicts a flow analysis of cells transfected with the dual promoter construct depicted in FIG. 7A, a construct encoding an anti-CD19 CAR, or control (mock).

FIG. 7C depicts a graph for normalized mCherry signal from co-cultured effector and target cells by Incucyte analysis.

FIG. 8A depicts a dual promoter lentiviral construct containing an inducible iSynPro promoter and IL2 minimal promoter operably linked to a nucleic acid encoding a GFP reporter gene; an insulator sequence; a constitutive EF1a promoter operably linked to a nucleic acid encoding an anti-CD19 CAR, a P2A sequence, a selectable DHFRdm protein, a T2A sequence, and a truncated EGFR (EGFRt) marker protein, and a BGH polyA sequence.

FIG. 8B depicts a flow analysis of cells transduced with the dual promoter lentiviral construct depicted in FIG. 8A and treated with PMA and ionomycin.

FIG. 9 depicts an exemplary embodiment of a system in which a nucleic acid encoding a dual promoter system includes a polynucleotide encoding an anti-CD19 CAR linked to a constitutive EF1 promoter, and a polynucleotide encoding a CA-STAT5a protein linked to an inducible promoter (iSynPro).

FIG. 10 depicts an exemplary embodiment of a system in which a nucleic acid encoding a dual promoter system includes a polynucleotide encoding an anti-CD19 CAR linked to a constitutive EF1. promoter, and a polynucleotide encoding a CA-STAT5aER fusion protein linked to an inducible promoter (iSynPro).

### DETAILED DESCRIPTION

Some embodiments of the methods and compositions provided herein relate to dual promoter systems including a nucleic acid comprising a polynucleotide comprising a promoter operably linked to a polynucleotide encoding a receptor, and a polynucleotide comprising an inducible promoter operably linked to a polynucleotide encoding a payload, wherein transcription from the inducible promoter is inducible by activation of the receptor. In some embodiments, the receptor is expressed in a host cell comprising the nucleic acid. In some embodiments, a ligand binds to the receptor, thereby activating the receptor which thereby induces transcription from the inducible promoter and expression of the payload. In some embodiments, the inducible promoter is quiescent in the absence of the ligand.

In some embodiments, transcription from the inducible promoter is further modulated by inhibiting a signal between the activated receptor and the inducible promoter. In some embodiments, the activity of the payload can be further modulated.

Some embodiments of the methods and compositions provided herein also include gene expression constructs that provide constitutive and/or inducible gene expression. In some embodiments, the gene expression constructs described herein provide constitutive and/or inducible gene expression in immune cells. In some embodiments, the gene expression constructs described herein provide constitutive and/or inducible gene expression in T cells. In some embodiments, the gene expression construct comprises, consists of, or consists essentially of a dual gene expression construct comprising dual gene expression cassettes.

One of the cassettes in these embodiments is a constitutive T cell gene expression cassette and the other cassette is an T cell activation inducible gene expression cassette. Presently, a gene expression construct comprising a dual promoter system (“dual promoter construct”) comprising a chimeric antigen receptor and/or T cell receptor (CAR/TCR) activation inducible promoter regulated gene expression cassette, as well as, an constitutive promoter (e.g., EF1alpha) regulated gene expression cassette does not exist. The dual promoter construct design provided herein combines two expression cassettes in a single dual promoter construct, which desirably avoids the need for co-electroporation of two constructs.

In some embodiments, a dual promoter construct comprises an inducible promoter in tandem with a constitutive promoter in a single nucleic acid. In some embodiments, the activation inducible promoter can be any promoter that is activated by activation of one or more receptors on the surface and/or within a cell and/or chemically. In some embodiments, the activation inducible promoter is a CAR/TCR inducible synthetic promoter (iSynPro), as described in the U.S. 2020/0095573 and WO 2018/213332, which are each expressly incorporated by reference in its entirety. In some embodiments, transcription from the iSynPro promoter is induced by activation of a T cell receptor (“TCR”) and/or a chimeric antigen receptor (“CAR”) on the surface of a T cell. In some embodiments, the inducible nature of iSynPro promoter that is activated by activation of a TCR and/or CAR on a T cell is useful, for example, in CAR T cell therapy, when the expression of a desired molecule in CAR T cells is selectively required in order to avoid a side effect that can occur when constitutive expression of CAR T cell occurs, such as cytokine storms. In some embodiments, a dual promoter construct described herein further comprises at least one insulator sequence. An “insulator” is a sequence that not only avoids “cross-talk” between the transcription factors acting on the inducible and constitutive promoters within the dual promoter construct but also insulates transcriptional activity of the dual promoter construct from the effects of a surrounding chromosomal environment. In some embodiments, the dual promoter construct further comprises a poly A tail sequence at the end of each expression cassette. In some embodiments, a poly A tail sequence at the end of each expression cassette provides a unique poly A tail sequence at the end of each transcript generated from each expression cassette. This feature avoids read-through from one transcript to the other during translation when the transcripts from the two gene expression cassettes are present on a single mRNA molecule.

In some embodiments, a T cell comprises a dual promoter construct that constitutively expresses a CAR and is capable of or configured for co-expressing a payload from the inducible promoter. The T cell is activated by a ligand that binds to the CAR and activates the CAR. The activation of the T cell results in the expression of the payload. The payload can include one or more therapeutic molecules that ameliorate, inhibit or treat a disorder in a subject. Such T cells can be used in clinical trials to assess the therapeutic potential of the cells for the amelioration, inhibition and/or treatment of a disorder in the subject. In some embodiments, such T cells can also be used for understanding the basic immunobiology research. For example, in some embodiments, such T cells can be used for understanding their interactions with other cells of the immune system.

Some embodiments of the methods and compositions provided herein can include aspects disclosed in U.S. 2020/0095573 entitled “GENERATING MAMMALIAN T CELL ACTIVATION INDUCIBLE SYNTHETIC PROMOTERS (SYN+PRO) TO IMPROVE T CELL THERAPY”; and WO 2020/047165 entitled “SELF-INACTIVATING-TRANSPOSASE PLASMIDS AND USES THEREOF”, which are each expressly incorporated by reference in its entirety.

### DEFINITIONS

“Conditional” or “Inducible” as used herein have their plain and ordinary meaning when read in light of the specification, and may include but is not limited to, for example, a nucleic acid construct that includes a promoter, which provides for gene expression in the presence of an inducer and does not substantially provide for gene expression in the absence of the inducer. Without being limiting, examples of inducible promoters for mammalian expression constructs include tetracycline, ecdysone, streptogramins, macrolides or doxycycline inducible promoters. Without being limiting, examples of inducible promoters for bacterial expression constructs include but are not limited to a T7 promoter, lac promoter, trc promoter, tac promoter, tetA promoter, araBAD promoter or a rhaPBAD promoters. Without being limiting, insect-derived promoters include but are not limited to pB2 and polyhedrin promoters. In some alternatives herein, a promoter is provided, wherein the promoter is an inducible promoter for mammalian protein expression. In some alternatives, the promoter is an inducible synthetic promoter. In some alternatives, the promoter is selected to be activated following CAR T cell activation.

A “promoter” has its plain and ordinary meaning when read in light of the specification, and may include but is not limited to, for example, a nucleotide sequence that directs the transcription of a structural gene. In some alternatives, a promoter is located in the 5′ non-coding region of a gene, proximal to the transcriptional start site of a structural gene. Sequence elements within promoters that function in the initiation of transcription are often characterized by consensus nucleotide sequences. It is a region of DNA that initiates transcription of a particular gene. Promoters are located near the transcription start sites of genes, on the same strand and upstream on the DNA (towards the 5′ region of the sense strand). Promoters can be about 100, 200, 300, 400, 500, 600, 700, 800, or 1000 base pairs long or within a range defined by any two of the aforementioned lengths. As used herein, a promoter can be constitutively active, repressible or inducible. If a promoter is an inducible promoter, then the rate of transcription increases in response to an inducing agent. In some alternatives, the promoter is a synthetic promoter.

“Transcription factor response elements,” have their plain and ordinary meaning when read in light of the specification, and may include but is not limited to, for example, short sequences of DNA within a gene promoter region that are able to bind specific transcription factors and regulate transcription of genes. Under conditions of stress, a transcription activator protein binds to the response element and stimulates transcription. They may also be a short (50-1500 bp) region of DNA that can be bound by proteins (activators) to increase or promote or enhance the likelihood that transcription of a particular gene will occur or the level of transcription that takes place. These activator proteins are usually referred to as transcription factors. Enhancers are generally cis-acting, located up to 1 Mbp (1,000,000 bp) away from the gene and can be upstream or downstream from the start site, and either in the forward or backward direction. An enhancer may be located upstream or downstream of the gene it regulates. A plurality of enhancer domains may be used in some embodiments to generate greater transcription e.g., multimerized activation binding domains can be used to further enhance or increase the level of transcription. Furthermore, an enhancer doesn't need to be located near the transcription initiation site to affect transcription, as some have been found located in several hundred thousand base pairs upstream or downstream of the start site. Enhancers do not act on the promoter region itself but are bound by activator proteins, These activator proteins interact with the mediator complex, which recruits polymerase II and the general transcription factors, which then begin transcribing the genes. Enhancers may also be found within introns. An enhancer's orientation may even be reversed without affecting its function. Additionally, an enhancer may be excised and inserted elsewhere in the chromosome, and still affect gene transcription. An example of an enhancer binding domain is the TCR. alpha enhancer. In some alternatives, the enhancer domain in the alternatives described herein is a TCR alpha enhancer.

“Transcriptional activator domains” or “Transcriptional activation domain” have their plain and ordinary meaning when read in light of the specification, and may include but is not limited to, for example, specific DNA sequences that can be bound by a transcription factor, in which the transcription factor can thereby control the rate of transcription of genetic information from DNA to messenger RNA. Specific transcription factors can include but is not limited to SP1, AP1, C/EBP, heat shock factor, ATFCREB, c-Myc, Oct-1 and/or NF-1.

A “chimeric antigen receptor” (CAR) described herein, also known as chimeric T-cell receptor, has its plain and ordinary meaning when read in light of the specification, and may include but is not limited to, for example, an artificial T-cell receptor or a genetically engineered receptor, which grafts a desired specificity onto an immune effector cell. A CAR may be a synthetically designed receptor comprising a ligand binding domain of an antibody or other protein sequence that binds to a molecule associated with the disease or disorder and is linked via a spacer domain to one or more intracellular signaling domains of a T-cell or other receptors, such as a costimulatory domain. In some alternatives, a cell, such as a mammalian cell, is manufactured, wherein the cell comprises a chimeric antigen receptor. These receptors can be used to graft the specificity of a monoclonal antibody or a binding portion thereof onto a T-cell, for example. In some alternatives herein, the genetically engineered cell further comprises a sequence that encodes a chimeric antigen receptor. In some alternatives, the chimeric antigen receptor is specific for a molecule on a tumor cell. A chimeric antigen receptor or an engineered cell expressing a T cell receptor can be used to target a specific tissue.

“Ligand” as described herein, refers to a substance that can form a complex with a biomolecule. By way of example and not of limitation, ligands can include substrates, proteins, small molecules, inhibitors, activators, nucleic acids and neurotransmitters. Binding can occur through intermolecular forces, for example ionic bonds, hydrogen bonds, and van der walls interactions. Ligand binding to a receptor protein can alter the three-dimensional structure and determine its functional state. The strength of binding of a ligand is referred to as the binding affinity and can be determined by direct interactions and solvent effects. A ligand can be bound by a “ligand binding domain.” A ligand binding domain, for example, can refer to a conserved sequence in a structure that can bind a specific ligand or a specific epitope on a protein. The ligand binding domain or ligand binding portion can comprise an antibody or binding fragment thereof or scFv, a receptor ligand or mutants thereof, peptide, and/or polypeptide affinity molecule or binding partner. Without being limiting, a ligand binding domain can be a specific protein domain or an epitope on a protein that is specific for a ligand or ligands.

“PMA” or “phorbol 12-myristate 13-acetate” is a diester of phorbol and a potent tumor promoter often employed in biomedical research to activate the signal transduction enzyme protein kinase C (PKC). In the alternatives, herein, PMA is used to induce an inducible synthetic promoter.

“Ionomycin” is an ionophore produced by the bacterium Streptomyces conglobatus. It is used in research to raise the intracellular level of calcium (Ca2+) and as a research tool to understand Ca2+ transport across biological membranes. It is also used to stimulate the intracellular production of the following cytokines; interferon, perforin, IL-2, and/or IL-4—usually in conjunction with PMA. In the alternatives herein, the ionomycin is used to induce an inducible synthetic promoter.

A “minimal promoter” is used to get a low amount of transcription of a target gene. They have key sequences to specify the transcription start site, but only weakly activates transcription because it does not recruit RNA Polymerase or transcription factors strongly. In the alternatives herein, the minimal promoter sequence is an IL2-minimal promoter sequence which is a fragment of IL2 promoter (−70 to +47) containing a TATA box. Prior to construction and use of an IL-2 minimal promoter, a commercial minimal promoter from an inducible gene expression construct was tested. The commercially available minimal promoter was shown to work in cell lines but not in primary T cells. “Subject” or “patient,” as described herein, refers to any organism upon which the alternatives described herein may be used or administered, for experimental, diagnostic, prophylactic, and/or therapeutic purposes. Subjects or patients include, for example, animals. In some alternatives, the subject is mice, rats, rabbits, non-human primates, and/or humans. In some alternatives, the subject is a cow, sheep, pig, horse, dog, cat, primate or a human.

“Cytokines” as described herein, refers to small proteins (5-25 kDa) that are important in cell signaling. Cytokines are released by cells and affect the behavior of other cells, and sometimes the releasing cell itself, such as a T-cell. Cytokines can include, for example, chemokines, interferons, interleukins, lymphokines, and/or tumor necrosis factor. Cytokines can be produced by a broad range of cells, which can include, for example, immune cells like macrophages, B lymphocytes, T lymphocytes and/or mast cells, as well as, endothelial cells, fibroblasts, and/or various stromal cells.

“Vector,” “Expression vector” or “construct” is a nucleic acid used to introduce heterologous nucleic acids into a cell that has regulatory elements to provide expression of the heterologous nucleic acids in the cell. Vectors include but are not limited to plasmid, minicircles, yeast, and/or viral genomes. In some alternatives, the vectors are plasmid, minicircles, yeast, or viral genomes. In some alternatives, the vector is a viral vector. In some alternatives, the viral vector is a lentivirus. In some alternatives, the vector is for protein expression in a bacterial system such as E. coli. In some alternatives, the vector is a lentiviral vector. In some alternatives, the vector is a foamy viral vector, adenoviral vectors, retroviral vectors or lentiviral vectors. In some alternatives, the vector is for protein expression in a bacterial system, such as E. coli. In some alternatives, wherein the vector is a lentiviral vector, a transposase based minicircle or a nanoplasmid.

An “insulator” can include a nucleotide sequence that blocks the interaction between enhancers and promoters such as an insulator having enhancer blocking activity, and/or insulate the transcriptional activity of a nucleic acid inserted into a genome from surrounding chromosomal effects, such as repressive effects against transcription of the inserted nucleic acid.

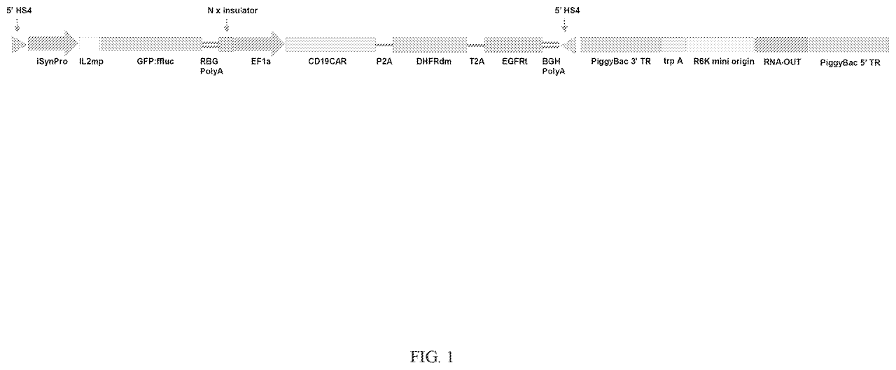

Tamoxifen, CAS RN: 10540-29-1, is also known as 2-(4((1Z)-1,2-diphenyl-1-butenyl)phenoxy)-N,N-dimethyl-ethanamine, or (Z)-2-(para-(1,2-Diphenyl-1-butenyl)phenoxy)-N,N-dimethylamine (IUPAC), and has a molecular formula of C₂₆H₂₉NO, M. W. 371.52. Tamoxifen is a Selective Estrogen Receptor Modulator with tissue-specific activities. Tamoxifen acts as an anti-estrogen (inhibiting agent) agent in the mammary tissue, but as an estrogen (stimulating agent) in cholesterol metabolism, bone density, and cell proliferation in the endometrium. Tamoxifen is frequently administered orally as a pharmaceutically acceptable salt. For example, Tamoxifen citrate (RN 54965-24-1, M. W. 563.643) is indicated for treatment of meta.static breast cancer, and as an adjuvant for the treatment of breast cancer in women following mastectomy axillary dissection, and breast irradiation. Tamoxifen citrate is also indicated to reduce incidence of breast cancer in women at high risk for breast cancer. Metabolites of tamoxifen in rat, mouse and human breast cancer patients, including major metabolites N-desmethyltamoxifen (RN 31750-48-8, M. W. 357.494) and 4-hydroxytamoxifen (4-OHT) (RN 68392-35-8, M. W. 387.52, Afimoxifene), are disclosed in Robinson et al., Metabolites, pharmacodynamics, and pharmacokinetics of tamoxifen in rats and mice compared to the breast cancer patient. Drug Metab Dispos January 1991 19:36-43, whith is incorporated by reference herein in its entirety. Additional cytochrome P-450 metabolites, which can be used with any one or more of the embodiments disclosed herein, are disclosed in Crewe et al., 2002, including cis-4-hydroxytamoxifen (RN 174592, M. W. 387.52; Afimoxifene, E-isotner), and 4′-hydroxytamoxifen ((Z)-4-(1-(4-(2-(dimethylamino)ethoxy)phenyl)-1-phenylbut-1-en-2-yl)phenol). See Crewe et al., 2002. Metabolism of Tamoxifen by recombinant human cytochrome P-450 enzymes: Formation of the 4-hydroxy, 4′-hydroxy and N-desmethyl metabolites and isomerization of trans-4-hydroxytamoxifen, Drug Metab Dispos, 30(8): 869-874, FIG. 1, which is incorporated herein by reference. Compounds with structural similarity to tamoxifen, which can be used with any one or more of the embodiments disclosed herein, include, but are not limited to, cis-tamoxifen (RN 13002-65-8, M. W. 371.521), 4-methyltamoxifen (RN 73717-95-5, M. W. 385.548), N-desmethyltamoxifen (RN 31750-48-8, M. W. 357.494), (Z)-desethyl methyl tamoxifen (RN 15917-50-7, M. W. 357.494), (E)-desethyl methyl tamoxifen (RN 31750-45-5, M. W. 357.494), trans-4-hydoxytamoxifen (RN 68047-06-3, M. W. 387.52), Afimoxifene (RN 68392-35-8, M. W. 387.52, 4-hydroxytamoxifen), Afimoxifene, E-isomer (RN 174592-47-3, M. W. 387.52), 4-chlorotamoxifen (RN 77588-46-6, M. W. 405.966), 4-fluorotamoxifen (RN 73617-96-6, M. W. 389.511), Toremifene (RN 89778-26-7, M. W. 405.966), desethyl tamoxifen (RN 19957-51-8, M. W. 343.47), (E)-desethyl tamoxifen (RN 97151-10-5, M. W. 343.47), (Z)-desethyl tamoxifen (RN 97151-11-6, M. W. 343.47), Miproxifene (RN 129612-87-9, M. W. 429.6), 2-(p-(beta-ethyl-alpha-phenylstyryl)phenoxy)triethylamine (RN 749-86-0, M. W. 399.575), Droloxifene (RN 82413-20-5, M. W. 387.52), 4-iodo-tamoxifen (RN 116057-68-2, M. W. 497.413), dihydrotamoxifen (RN 109640-20-2, M. W. 373.537), (E)-N,N-dimethyl-2-(4-(1-(2-methylphenyl)-2-phenyl-1-butenyl)phenoxy)ethanamine (RN 97150-96-4, M. W. 385.548), or 4-hydroxytoremifene (RN 110503-62-3, M. W. 421.965); and/or pharmaceutically acceptable salts and/or hydrates or solvates thereof. For example, citrate salts of tamoxifen, or citrate salts of compounds with structural similarity to tamoxifen, include, but are not limited to tamoxifen citrate (RN 54965-24-1, M. W. 563.64), 2-(p-(1,2-diphenyl-1-butenyl)phenoxy)-N,N-dimethylethylamine citrate (RN 7244-97-5, 563.64), (E)-tamoxifen citrate (RN 76487-65-5, M. W. 563.64), Toremifene citrate (RN 89778-27-8, M. W. 598.088), Droloxifene citrate (RN 97752-20-0, M. W. 579.64), 2-(p-(1,2-bis(p-methoxyphenyl)-1-butenyl)phenoxy)triethylamine citrate (RN 42920-39-8, M. W. 651.748), 2-(4-(1,2-diphenylethenyl)phenoxy)-N,N-diethyl-ethanamine 2-hydroxy-1,2,3-propanetricarboxylate (RN 40297-42-5, M. W. 563.643), 2-(p-(alpha-phenylstyryl)phenoxy)triethylamine citrate (RN 102433-95-4, M. W. 563.64), 2-(p-(2-(p-methoxyphenyl)-1-phenyl-1-butenyl)phenoxy)triethylamine citrate (1:1) (RN 42824-34-0, M. W. 637.72), 2-(p-(1-(p-methoxyphenyl)-2-phenylpropenyl)phenoxy)triethylamine citrate (RN 13554-24-0, M. W. 607.696), 2-(p-(alpha-(p-methoxyphenyl)styryl)phenoxy)triethylamine citrate monohydrate (RN 13542-71-7, M. W. 593.669), 2-(p-(p-methoxy-alpha-phenylphenethyl) phenoxy)triethylamine citrate (RN 16421-72-0, M. W. 595.685), alpha-(p-(2-(diethylamino)ethoxy)phenyl)-beta-ethyl-p-methoxy-alpha-phenylphenethyl alcohol citrate (1:1) (RN 35263-93-5, M. W. 639.737), 1-(p-(2-(diethylamino)ethoxy)phenyl)-2-(p-methoxyphenyl)-1-phenylethanol citrate (M. W. 611.68), alpha-p-(2-(diethylamino)ethoxy)phenyl)-beta-ethyl-alpha-(p-hydroxyphenyl)-p-methoxyphenethyl alcohol citrate (RN 35263-96-8, M. W. 655.737), and/or 2-(p-(p-methoxy-alpha-methylphenethyl)phenoxy)-triethyl amine citrate (RN 15624-34-7, M. W. 533.614).

### Certain Nucleic Acids

Some embodiments of the methods and compositions provided herein include nucleic acids encoding a dual promoter system. Some such embodiments include a nucleic acid comprising a first polynucleotide comprising an inducible first promoter operably linked to a polynucleotide encoding a payload, wherein transcription from the first promoter is inducible by activation of a receptor. In some embodiments, the nucleic acid also includes a second polynucleotide comprising a second promoter operably linked to a polynucleotide encoding the receptor. In some embodiments, the first polynucleotide can be 5′ or 3′ of the second polynucleotide. In some embodiments, the nucleic acid also includes a first insulator located between the first polynucleotide and the second polynucleotide. An example of an embodiment is depicted in FIG. 1.

In some embodiments, the first promoter comprises a synthetic inducible promoter. In some embodiments the first promoter comprises a nucleotide sequence having a percentage identity with the nucleotide sequence set forth in any one of SEQ ID NOs:1-5 of at least 90%, 91%, 92, 93%, 94%, 95%, 96%, 97%, 98%, 99% or 100% or a percentage of sequence identity that is within a range defined by any two of the aforementioned percentages. In some embodiments, the first promoter comprises the nucleotide sequence set forth in SEQ ID NO:1. In some embodiments, the first promoter also includes a minimal promoter. In some embodiments, the minimal promoter comprises an IL-2. minimal promoter. Aspects useful with embodiments provided herein are disclosed in U.S. 2020/0095573, which is expressly incorporated by reference in its entirety.

In some embodiments, the second promoter is constitutive. Examples of constitutive promoters useful with embodiments provided herein include an EF1α promoter, a PGK promoter, a MND promoter, a MSCV promoter, a CMV promoter, a UBC promoter, a CAGE promoter, or an SV40 promoter. In some embodiments, the second promoter comprises an EF1α promoter.

In some embodiments, the nucleic acid includes a first insulator located between the first polynucleotide and the second polynucleotide. In some such embodiments, the first insulator comprises an enhancer-blocking insulator. Examples of insulators useful with embodiments provided herein include a 3′ HS-A β-globin insulator, an 5′ HS4 β-globin insulator, an 5′ HS5 insulator, an scs insulator, an scs' insulator, a gypsy insulator, a CTCF insulator, a scaffold attachment region (SAR) insulator, an DMD/ICR insulator, an apoB insulator, a DM1 insulator, a BEAD-1 insulator, a HS2-6 insulator, a DMD/ICR insulator, a Lys 5′ A insulator, an RO insulator, a UR1 insulator, an sns insulator, a Fab-7 insulator, a Fab-8 insulator, a faswb insulator, an eve promoter insulator, an HMR tRNAThr insulator, a Chal UAS insulator, an UASrpg insulator, or a STAR insulator. In some embodiments, the first insulator comprises a 3′ HS-A β-globin insulator. In some embodiments the first insulator comprises a nucleotide sequence having a percentage identity with the nucleotide sequence set forth in SEQ ID NO:6 of at least 90%, 91%, 92, 93%, 94%, 95%, 96%, 97%, 98%, 99% or 100% or a percentage of sequence identity that is within a range defined by any two of the aforementioned percentages. In some embodiments, the first insulator comprises the nucleotide sequence set forth in SEQ ID NO:6.

In some embodiments, the receptor is activated by the receptor binding a ligand for the receptor. In some such embodiments, the activated receptor can induce transcription from the first promoter via a signal. In some embodiments, the signal comprises a pathway of one or more activated components, for example, the activated receptor activates other components. Examples of activated components include phosphorylated proteins. In some embodiments, the activated receptor binds directly to the first promoter to induce transcription from the first promoter.

In some embodiments, the receptor comprises an intracellular receptor, such as a nuclear receptor. In some embodiments, the receptor comprises a cell surface receptor. In some embodiments, the receptor comprises a cell receptor (TCR). In some embodiments, the receptor comprises a chimeric antigen receptor (CAR). In some embodiments, the CAR comprises an intracellular signaling domain comprising a costimulatory domain selected from the group consisting of CD27, CD28, 4-1BB, OX-40, CD30, CD40, PD-1, ICOS, LEA-1, CD2, CD7, NKG2C, and B7-H3, preferably in combination with a CD3-zeta domain or a functional portion thereof. In some embodiments, the intracellular signaling domain comprises a 4-1BIS costimulatory domain. In some embodiments, the intracellular signaling domain comprises a CD28 cytoplasmic domain. Aspects useful with embodiments provided herein are disclosed in U.S. 2018/0009891 which is expressly incorporated by reference in its entirety

In some embodiments, the second polynucleotide comprises a polynucleotide encoding a selectable marker, In some embodiments, the selectable marker comprises a cell-surface selectable marker, Examples of selectable markers useful with embodiments provided herein include a truncated EGFR (EGFRt) polypeptide or a truncated Her2 (Her2t) polypeptide. In some embodiments, the selectable marker comprises a drug selectable marker. In some embodiments, the drug selectable marker comprises a dihydrofolate reductase (DHFR) polypeptide. In some such embodiments, the DHFR can include a double mutant DHFR (dmDHFR), which includes two substitutions with regard to a wild type DHFR protein. Aspects useful with embodiments provided herein are disclosed in U.S. 2017/0267742 and U.S. 2017/0029774, which are each expressly incorporated by reference in its entirety.

Some embodiments also include a ribosome skip sequence or an internal ribosome entry site (IRES). In some embodiments, the ribosome skip sequence or IRES is located between the polynucleotide encoding the receptor and the polynucleotide encoding a selectable marker. In some embodiments, the ribosome skip sequence is selected from the group consisting of a P2A sequence, a T2A. sequence, an E2A sequence, and an F2A sequence.

In some embodiments, the first polynucleotide and the second polynucleotide are located between a second insulator and a third insulator. In some such embodiments, the second insulator and/or third insulator comprises an enhancer-blocking insulator. Examples of insulators useful with embodiments provided herein include a 3′ HS-A β-globin insulator, an 5′ HS4-βglobin insulator, an 5′ HS5 insulator, an scs insulator, an scs' insulator, a gypsy insulator, a CTCF insulator, a scaffold attachment region (SAR) insulator, an DMD/ICR insulator, an apoB insulator, a DM1 insulator, a BEAD-1 insulator, a HS2-6 insulator, a DMD/ICR insulator, a Lys 5′ A insulator, an RO insulator, a UR1 insulator, an sns insulator, a Fab-7 insulator, a Fab-8 insulator, a faswb insulator, an eve promoter insulator, an HMR tRNAThr insulator, a Chal LAS insulator, an UASrpg insulator, or a STAR insulator. In some embodiments, the second insulator and/or third insulator comprises a 5′ HS4 β-globin insulator.

In some embodiments, the first polynucleotide and/or second polynucleotide comprises a poly A tail.

In some embodiments, the payload comprises a first protein or a first non-coding RNA. In some embodiments, the payload comprises a second protein or a second non-coding RNA. In some embodiments, a ribosome skip sequence or an internal ribosome entry site (IRES) is located between a polynucleotide encoding the first protein or the first non-coding RNA and a polynucleotide encoding the second protein or the second non-coding RNA.

In some embodiments, the payload comprises an intracellular protein, a transmembrane protein, a membrane bound protein, a secreted protein, or a microRNA (miRNA). In some embodiments, the payload comprises a therapeutic protein or non-coding RNA. In some embodiments, a target for the payload is intracellular, extracellular, of another cell. Examples of payloads useful with embodiments provided herein include cytokines, hormones, antigen-binding proteins such as antibodies, fusion proteins, or receptors, such as CARs. In some embodiments, the payload comprises a signal transducer and activator of transcription (STAT) protein, or a functional derivative of a STAT protein. In some embodiments, the STAT protein comprises a constitutively activated STAT (CA-STAT) protein. In some embodiments, the STAT protein comprises a STAT5a protein. In some embodiments, the STAT protein comprises a fusion protein comprising a functional portion of a constitutively activated STAT5a and of an estrogen receptor.

Some embodiments of the methods and compositions provided herein include vectors comprising any one of the nucleic acids encoding a dual promoter system provided herein. In some embodiments, the vector comprises a transposon vector, a viral vector, a plasmid, a nanoplasmid, or a minicircle. In some embodiments, the vector comprises a lentiviral vector. In some embodiments, the vector comprises a transposon sequence. In some embodiments, the transposon sequence comprises a PiggyBac transposon sequence, or a Sleeping Beauty transposon sequence. For example, a PiggyBac transposon system can include transposon-specific inverted terminal repeats (ITRs) and a transposase. A PiggyBac transposon system enables one or more genes of interest located between two ITRs to be easily and efficiently mobilized into and out of target genomes. Thus, in some embodiments, the transposase can recognize the ITRs comprised within the dual promoter construct and efficiently catalyze the positioning of the dual promoter construct within a chromosome or an extrachromosomal element. Some embodiments include a vector comprising a polynucleotide sequence encoding a self-inactivating transposase. Examples of such self-inactivating transposases include a self-inactivating transposase PiggyBac (sinPB) transposase or a self-inactivating transposase Sleeping Beauty (sinSB) transposase. Aspects useful with embodiments provided herein are disclosed in WO 2020/047165 and U.S. 2017/0029774, which are each expressly incorporated by reference in its entirety.

Some embodiments of the methods and compositions provided herein include pharmaceutical compositions comprising any one of the nucleic acids encoding a dual promoter system provided herein and a pharmaceutically acceptable excipient.

TABLE 1 lists certain sequences useful with embodiments provided herein

TABLE 1

| SEQ ID NO. | |
|---|---|
| Features | Sequence |
| SEQ ID NO: 01 | TCGAATGAGTCACATCGATCTCCGCCCCCTCTTCGAGGGGGCG |
| (S1-61) | GGGTCGAGGAGGAAAAACTCGAATGAGTCACATCGACCCTTT |
| | GATCTTCGAGGGGACTTTCCGGGGTGGAGCAAGCGTGACAAG |
| | TCCACGTATGACCCGACCGACGATATCGAAGCCTACGCGCTG |
| | AACGCCAGCCCCGATCGACCCCGCCCCCTCGATTTCCAAGAAA |
| | TCGAATGACATCATCTTTCGAATGACATCATCTTTCGAGGGGA |
| | CTTTCCTCGAACTTCCTTCGAGGGGACTTTCCTCGAGGGGACT |
| | TTCCTCGAGGAGGAAAAACTCGAGTAGAGTCTAGA |
| SEQ ID NO: 02 | CTGCTTAGGGTTAGGCGTTTTGCGCTGCTTCGCGATCGAATGA |
| (expanded S1-61) | GTCACATCGATCTCCGCCCCCTCTTCGAGGGGGCGGGGTCGAG |
| | GAGGAAAAACTCGAATGAGTCACATCGACCCTTTGATCTTCGA |
| | GGGGACTTTCCGGGGTGGAGCAAGCGTGACAAGTCCACGTAT |
| | GACCCGACCGACGATATCGAAGCCTACGCGCTGAACGCCAGC |
| | CCCGATCGACCCCGCCCCCTCGATTTCCAAGAAATCGAATGAC |
| | ATCATCTTTCGAATGACATCATCTTTCGAGGGGACTTTCCTCG |
| | AACTTCCTTCGAGGGGACTTTCCTCGAGGGGACTTTCCTCGAG |
| | GAGGAAAAACTCGAGTAGAGTCTAGACTCTACATTTTGACAC |
| | CCCCAT |
| SEQ ID NO: 03 | CTGCTTAGGGTTAGGCGTTTTGCGCTGCTTCGCGATCGAAGAT |
| (S1-15) | CAAAGGGTCGATTTCTTGGAAATCGATGTGACTCATTCGATCA |
| | CGTCCTCGAGGAGGAAAAACTCGAGGAAAGTCCCCTCGAACT |
| | TCCTTCGAGGGGGCGGGGTCGAATGAGTCACATCGAGGAAAG |
| | TCCCCTCGAGGGGACTTTCCTCGATTTCTTGGAAATCGAAGAG |
| | GGGGCGGAGATCGAGTTTTTCCTCCTCGAGGAAAGTCCCCTCG |
| | ATCGACTCTACATTTTGACACCCCCAT |
| SEQ ID NO: 04 | CTGCTTAGGGTTAGGCGTTTTGCGCTGCTTCGCGATCGAGGAA |
| (S1-71) | AGTCCCCTCGAGGAAAGTCCCCTCGATTTCTTGGAAATCGAAT |
| | GACATCATCTTTCGATCACGTCCTCGAGGAAAGTCCCCTCGAG |
| | TTTTTCCTCCTCGAGGGGACTTTCCTCGATTTCCAAGAAATCG |
| | ATTTCTTGGAAATCGACCCTTTGATCTTCGAGGAGGAAAAACT |
| | CGAGTAGAGTCTAGACTCTACATTTTGACACCCCCAT |
| SEQ ID NO: 05 | CTGCTTAGGGTTAGGCGTTTTGCGCTGCTTCGCGATCGAGGAG |
| (S1-16) | GAAAAACTCGATTTCTTGGAAATCGAGGGGACTTTCCTCGAAA |
| | GATGATGTCATTCGAAGATCAAAGGGTCGATGTGACTCATTCG |
| | AGGGGACTTTCCTCGAGGGGGCGGGGTCGAATGACATCATCT |
| | TTCGAGGGGACTTTCCTCGAGGGGACTTTCCTCGAATGACATC |
| | ATCTTTCGATTTCTTGGAAATCGAGTAGAGTCTAGACTCTACA |
| | TTTTGACACCCCCAT |
| SEQ ID NO: 06 | TGAGATCTTTCACAAAACACCAGTTATGC |
| HS-A insulator | |

### Certain Host Cells

Some embodiments of the methods and compositions provided herein include a host cell comprising any one of the nucleic acids encoding a dual promoter system provided herein. In some embodiments, the host cell is an immune cell. In some embodiments, the host cell is a precursor T cell, or a hematopoietic stem cell. In some embodiments, the host cell is a T cell, a B cell, a natural killer cell, an antigen presenting cell, a dendritic cell, a macrophage, or a granulocyte such as a basophil, an eosinophil, a neutrophil, or a mast cell.

In some embodiments, the host cell is a CD4+ T cell or a CD8+ T cell. In some embodiments, the host cell is a CD8+ cytotoxic T cell selected from the group consisting of a naïve CD8+ Tcell, a CD8+ memory T cell, a central memory CD8+ T cell, a regulatory CD8+ T cell, an iPS derived CD8+ T cell, an effector memory CD8+ T cell, and a bulk CD8+ T cell. In some embodiments, the host cell is a CD4+ T helper cell selected from the group consisting of a naïve CD4+ T cell, a CD4+ memory T cell, a central memory CD4+ T cell, a regulatory CD4+ T cell, an iPS derived CD4+ T cell, an effector memory CD4+ T cell, and a bulk CD4+ T cell. In some embodiments, the host cell is allogenic to a subject, or is autologous to a subject. In some embodiments, the host cell is ex vivo. In some embodiments, the host cell is in vivo. In some embodiments, the host cell is mammalian. In some embodiments, the host cell is human.

Some embodiments of the methods and compositions provided herein include pharmaceutical compositions comprising any one of the host cells provided herein and a pharmaceutically acceptable excipient.

Some embodiments of the methods and compositions provided herein include preparing a host cell comprising any one of the nucleic acids encoding a dual promoter system provided herein. Some such embodiments include introducing into a host cell any one of the nucleic acids encoding a dual promoter system provided herein, or any one of the vectors comprising a nucleic acid encoding a dual promoter system provided herein. Some embodiments also include stimulating the host cell with an anti-CD3 and/or anti-CD28 antibody. Some embodiments also include expanding the host cell to obtain a population comprising a plurality of the host cell. Some such embodiments include culturing the population of host cell in the presence of certain cytokines, such as IL-7, IL-15 or IL-21 or any combination thereof. Some embodiments include selecting for a host cell. Some such embodiments include selecting for a drug selectable marker expressed by the host cell, such as DHFR using methotrexate. Some such embodiments include selecting for a cell surface selectable marker expressed by the host cell, such as an EGFRt polypeptide or Her2t polypeptide. Aspects useful with embodiments provided herein are disclosed in U.S. 2017/0015746, which is expressly incorporated by reference in its entirety.

### Certain Therapies and Methods

Some embodiments of the methods and compositions provided herein include methods of treating, ameliorating, or inhibiting, a disorder in a subject. Some embodiments include administering any one of the host cells provided herein to the subject in need thereof. Some embodiments include administering any one of the vectors provided herein to the subject in need thereof. In some embodiments, the subject is mammalian. In some embodiments, the subject is human.

Some embodiments also include reducing transcription from the first promoter by administering an inhibitor to the subject. In some such embodiments, the inhibitor blocks an intracellular signal between the activated receptor and the first promoter, thereby inhibiting or reducing transcription from the first promoter. In some embodiments, the inhibitor comprises a tyrosine kinase inhibitor. In some embodiments, the inhibitor comprises dasatinib.

Some embodiments also include increasing transcription from the first promoter by administering an activator to the subject. In some embodiments, the activator induces transcription from the first promoter. In some embodiments, the activator comprises a tyrosine kinase activator. In some embodiments, the activator is selected from phorbol 12-myristate 13-acetate (PMA) or lonomycin.

In some embodiments, the disorder is selected from a cancer, an inflammatory disorder, a viral infection, a fungal infection, and a bacterial infection, In some embodiments, the disorder comprises a cancer comprising a leukemia, a breast cancer, a stomach cancer, an esophageal cancer, a brain cancer, a uterine cancer, a prostate cancer, a bone cancer, a liver cancer, a pancreatic cancer, an ovarian cancer, a lung cancer, a colon cancer, a kidney cancer, a bladder cancer, or a thyroid cancer or any combination thereof

In some embodiments, the receptor comprises a chimeric antigen receptor (CAR). In some embodiments, the CAR specifically binds to an antigen selected from an antigen expressed on the surface of a cancer cell, a viral antigen, a fungal antigen, or a bacterial antigen. In some embodiments, the CAR specifically binds to an antigen selected from CD19 and CD171.

Some embodiments of the methods and compositions provided herein include methods for modulating activity of a host cell. Some such embodiments include providing any one of the host cells provided herein, wherein the receptor comprises a chimeric antigen receptor (CAR); and activating the CAR by contacting the CAR with a ligand, wherein the CAR specifically binds to the ligand, thereby inducing transcription from the first promoter.

Some embodiments also include reducing transcription from the first promoter by contacting the host cell with an inhibitor, wherein the inhibitor blocks an intracellular signal between the activated CAR and the first promoter, thereby inhibiting or reducing transcription from the first promoter. In some embodiments, the inhibitor comprises a tyrosine kinase inhibitor. In some embodiments, the inhibitor comprises dasatinib.

Some embodiments also include increasing transcription from the first promoter by contacting the host cell with an activator, wherein the activator induces transcription from the first promoter. In some embodiments, the activator comprises a tyrosine kinase activator. In some embodiments, the activator is selected from phorbol 12-myristate 13-acetate (MIA) or ionomycin.

Some embodiments also include inducing or increasing an activity of the payload by contacting the host cell with a payload activator. In some embodiments, the payload comprises a fusion protein comprising a functional portion of a constitutively activated signal transducer and activator of transcription 5a (CA-STAT5a) protein and of an estrogen receptor. In some embodiments, the payload activator comprises tamoxifen, an analogue of tamoxifen, a metabolite of tamoxifen, or derivative thereof.

### Certain Systems

Some embodiments of the methods and compositions provided herein include systems comprising any one of the host cells provided herein. In some such embodiments, the host cell comprises any one of the nucleic acids provided herein, and the receptor. In some embodiments, the host cell is an immune cell. In some embodiments, the host cell is allogenic to a subject, or is autologous to a subject. In some embodiments, the host cell is ex vivo. In some embodiments, the host cell is in vivo. In some embodiments, the host cell is mammalian. In some embodiments, the host cell is human.

In some embodiments, the receptor is activated. In some embodiments, the receptor comprises a chimeric antigen receptor (CAR).

Some embodiments also include an inhibitor which blocks an intracellular signal between the activated receptor and the first promoter, thereby reducing or inhibiting transcription from the first promoter. In some embodiments, the inhibitor comprises a tyrosine kinase inhibitor. In some embodiments, the inhibitor comprises dasatinib.

Some embodiments also include a payload activator, wherein the payload activator induces or increases an activity of the payload. In some embodiments, the payload comprises a fusion protein comprising a functional portion of a constitutively activated signal transducer and activator of transcription 5a (CA-STAT5a) protein and of an estrogen receptor. In some embodiments, the payload activator comprises tamoxifen, an analogue of tamoxifen, a metabolite of tamoxifen, or derivative thereof.

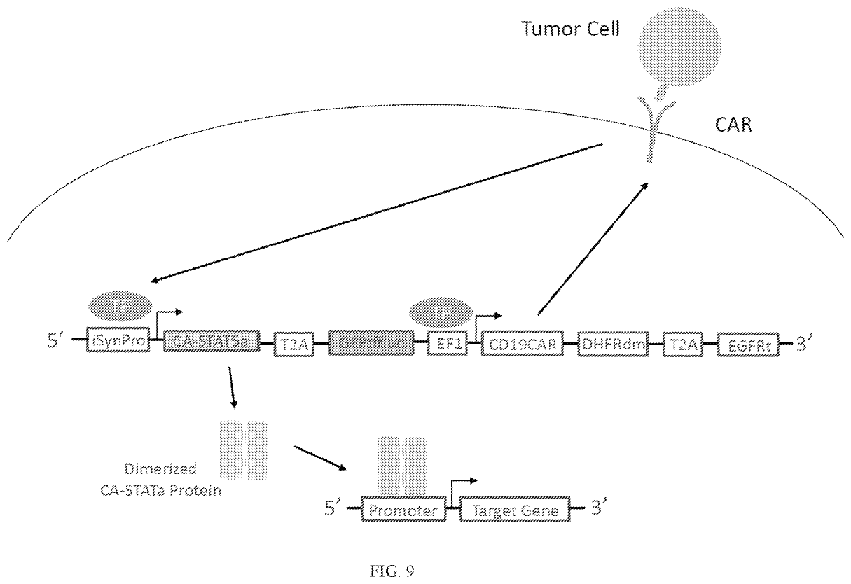

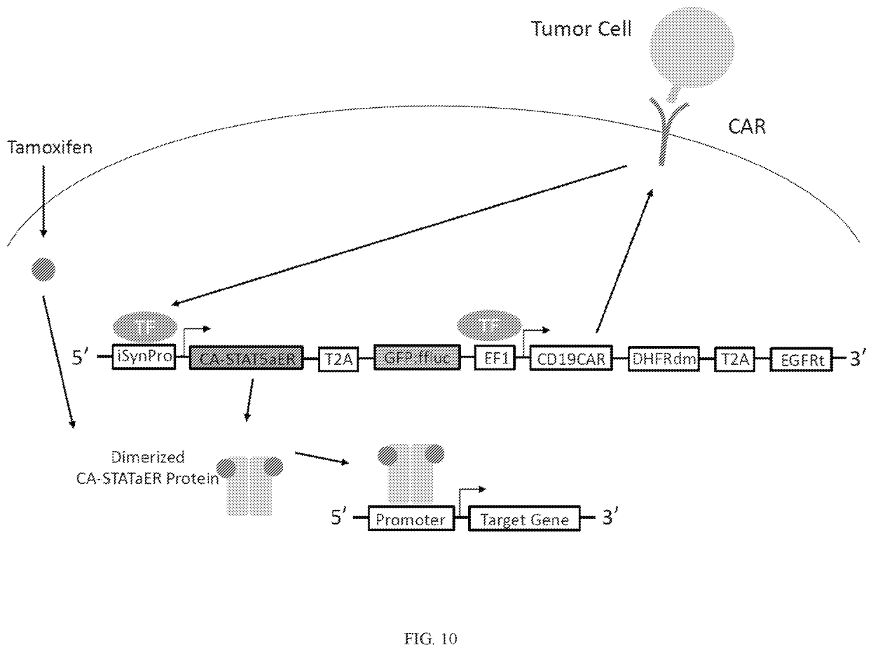

FIG. 9 depicts an example embodiment of a system in which a nucleic acid encoding a dual promoter system includes a polynucleotide encoding an anti-CD19 CAR linked to a constitutive EF1 promoter, and a polynucleotide encoding a CA-STAT5a protein linked to an inducible promoter (iSynPro). The host cell expresses the CAR, which is activated by a CD19 ligand on a tumor cell. The activated CAR induces transcription from the inducible promoter, which expresses CA-STAT5a, which induces transcription from a promoter linked to a target gene, such as a target gene endogenous to the host cell. In the absence of the CD19 ligand, transcription from the iSynPro promoter would be diminished and/or halted.

FIG. 10 depicts an example embodiment of a system in which a nucleic acid encoding a dual promoter system includes a polynucleotide encoding an anti-CD19 CAR linked to a constitutive EF1 promoter, and a polynucleotide encoding a CA-STAT5a protein linked to an inducible promoter (iSynPro). The host cell expresses the CAR which is activated by a CD19 ligand on a tumor cell. The activated CAR induces transcription from the inducible promoter, which expresses a fusion protein CA-STAT5aER. CA-STAT5aER may dimerize into an active form in the presence of tamoxifen. In the presence of tamoxifen, the CA-STATSaER dimerizes and induces transcription from a promoter linked to a target gene, such as a target gene endogenous to the host cell. In the absence of the CD19 ligand or tamoxifen, transcription from the promoter linked to the target gene would be diminished and/or halted.

### EXAMPLES

### Example 1—Preparation of a Dual Promoter Construct

A dual promoter construct was prepared using a nanoplasmid backbone (Nature Technology, Lincoln, NE) and other components. FIG. 1 depicts a schematic of the dual promoter construct which included a 5′ HSA insulator; an inducible synthetic promoter (iSynPro); an IL-2 minimal promoter (IL-2mp); a polynucleotide encoding a payload green fluorescent protein/luciferase protein (GFP:ffluc); an RBG poly A tail; a 3′ HS-A insulator (shown as ‘N×insulator’ which contained 2×3′HSA); a constitutive EF1α promoter (EF1a); a polynucleotide encoding an anti-CD19 chimeric antigen receptor (CD19CAR); a P2A ribosome skip sequence; a polynucleotide encoding a dihydrofolate reductase protein with two mutations (DHFRdm) to increase its efficiency as a selectable marker; a T2A ribosome skip sequence; a polynucleotide encoding a truncated EGFR (EGFRO polypeptide; a BGH poly A tail; a 5′ HSA insulator; a PiggyBac 3′ terminal repeat sequence; a trpA; R6K mini origin; an RNA-OUT; and a PiggyBac 5′ terminal repeat sequence.

### Example 2—Efficient Expression from an Induced Promoter in Activated Cells

Transfection of the single dual promoter construct, and of co-transduction of two lentiviral vectors containing either an EGFRt marker or a GFP marker was compared. The dual promoter construct was electroporated with a PiggyBac transposase mRNA into primary CD8+ T cells, Two lentiviral vectors containing either an EGFRt marker or a GFP marker were co-transduced into primary CD8+ T cells.

Transfected cells were stimulated with CTS Dynabeads anti-CD3/CD28. The cells were cultured in X-Vivo 15 media with KnockOut serum replacement, supplemented with 5 ng/ml IL-7. 0.5 ng/ml IL-15, and 10 ng/ml IL-21. Day 3 post-transfection, methotrexate selection (50 nM) was applied. On day 6, the beads were removed.

Cells were co-cultured with target CD19+ Tm-LCL cells to induce activation of the anti-CD19 CAR and induce transcription of GFP from the iSynPro promoter. After 24 hours, expression of EGFRt and GFP was measured by FACS analysis.

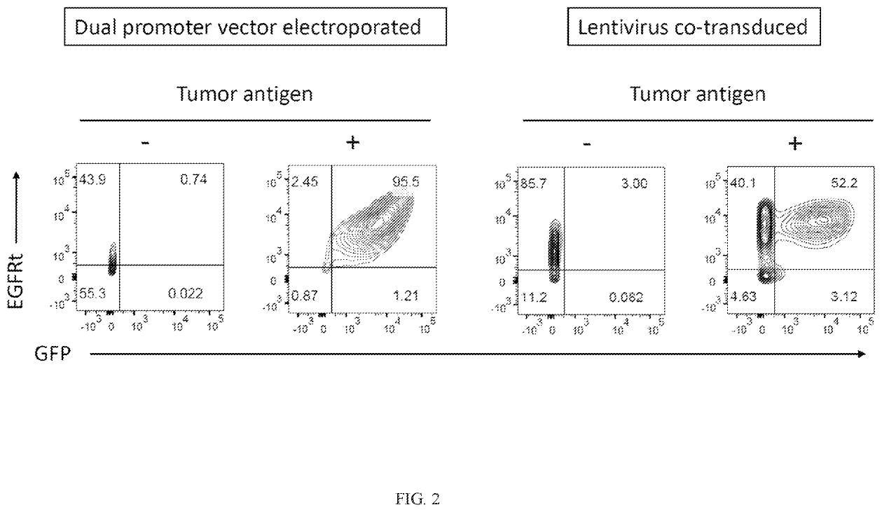

For cells transfected with the dual promoter construct: in the presence of CD19, EGFRt and GFP were both detected in about 95% of the population of cells (FIG. 2, left panel). For cells co-transduced with two lentiviral vectors containing either an EGFRt marker or a GFP marker: in the presence of CD19, EGFRt and GFP were both detected in about 52% of the population of cells, and EGFRt only was detected in about 40% of the population of cells (FIG. 2, right panel).

### Example 3—Inhibition of the Inducible Promoter in Activated CAR T Cells

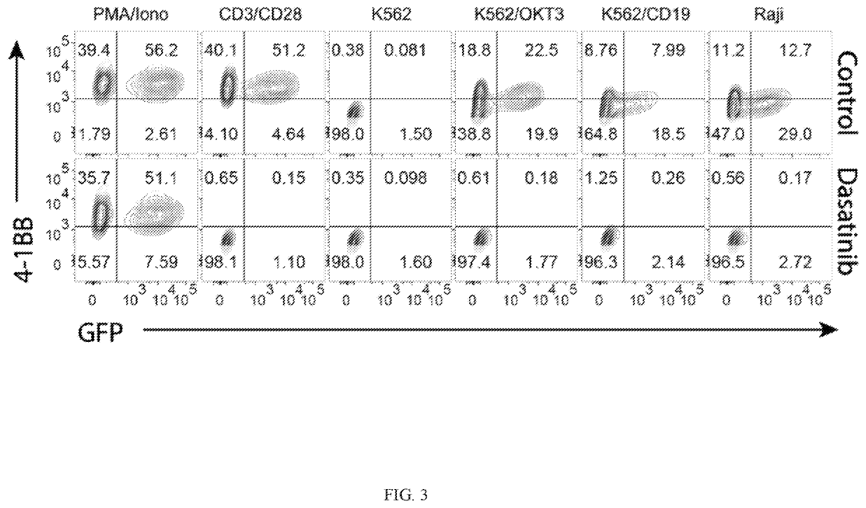

Inhibition of the iSynPro promoter was determined with the inhibitor, dasatinib. CD8⁺ T cells containing both an iSynPro-GFP:ffluc and an CD19CAR expression construct were prepared. The cells were treated under various conditions in the absence or presence of 100 nM dasatinib, including: (1) cultured in the presence phorbol 12-myristate 13-acetate (PMA) and Ionomycin which induced transcription from the iSynPro promoter; (2) cultured in the presence of antiCD3/CD28, which activates the T-cells; (3) co-cultured with K562 cells which do not express CD19; (4) co-cultured with CD19-K562/OKT3 cells, which activate the TCR; (5) co-cultured with CD19+ K562 cells, which activate the CD19 CAR; (6) co-cultured with CD19+ Raji cells, which activate the CD19 CAR. Expression of GFP from the iSynPro promoter, and expression of 41-BB, which was a result of the CD19 CAR activation were determined by FACS analysis. As shown in FIG. 3, dasatinib inhibited transcription from the iSynPro promoter in anti-CD19 T cells that had been otherwise activated by contacting with CD19 antigen.

### Example 4—In Vivo Characterization of the Dual Promoter Construct

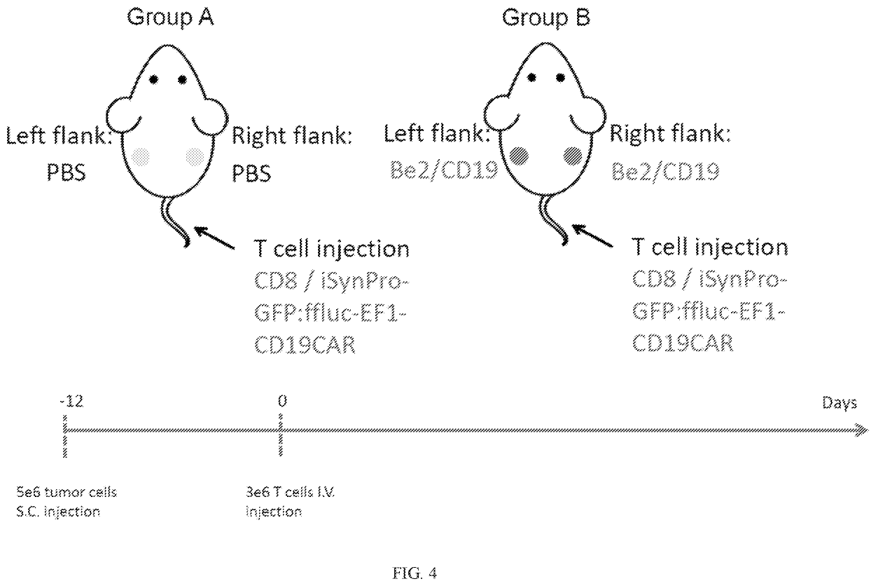

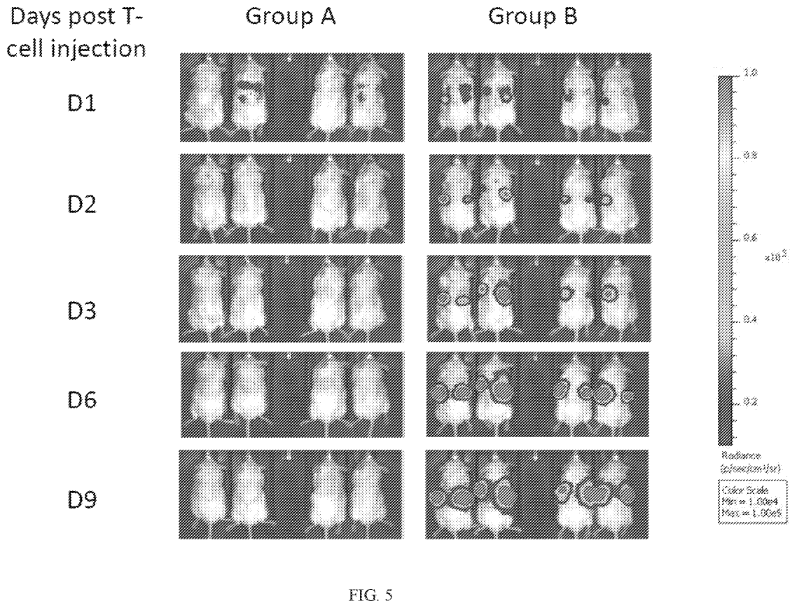

A xenograft model was used to characterize the dual promoter construct in vivo. The experimental design is depicted in FIG. 4. Briefly, mice were injected subcutaneously with 5×10⁶ CD19+ Be2 tumor cells at both flanks on day (-)12 (Group B). Control mice were injected with PBS on day (-)12 (Group A). CD8+ T cells containing the dual promoter construct were prepared. On day 0, 3×10⁶ of the CD8⁺ cells were injected through tail vein into the mice belonging to both Groups A and B. The mice were imaged from day 1 through day 9 (FIG. 5), and bioluminescence flux (measured in photons/sec) was measured.

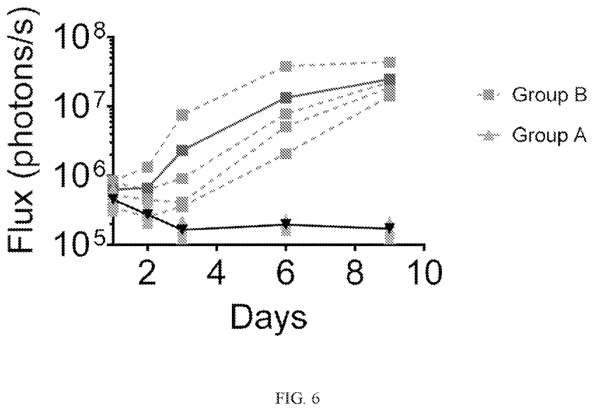

As shown in FIG. 5 and FIG. 6, Group A mice, which had been treated with T cells containing the dual promoter construct had substantially no luciferase expression. In contrast, Group B mice showed a significant luciferase signal, which increased with time indicating that the transcription from the iSynPro promoter had been induced in response to activation of the anti-CD19 CAR in vivo.

### Example 5—Dual Promoter Expression with an Inducible caSTAT5a Payload

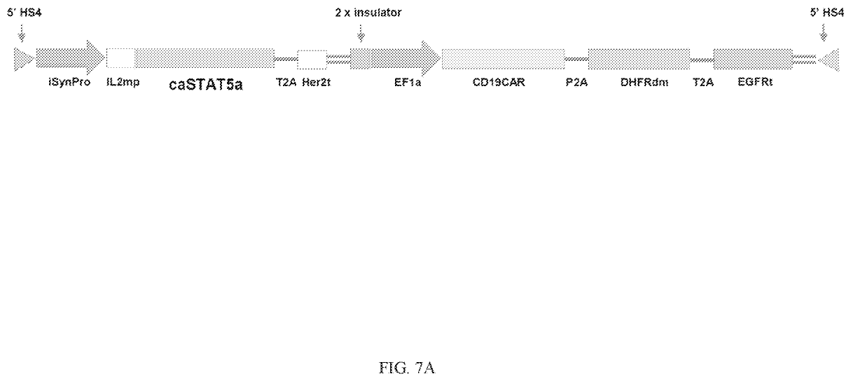

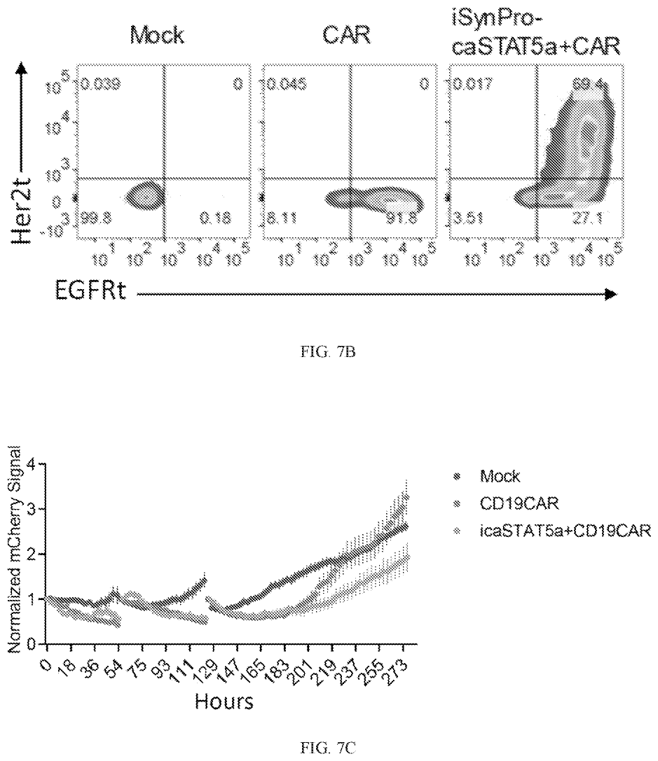

This example relates to a functional improvement of CAR T cells expressing a dual promoter piggyBac nanoplasmid (PBNP) in which an anti-CD19 CAR was constitutively expressed, and a constitutively active STAT5a (caSTAT5a) protein was inducible expressed. The dual promoter construct depicted in FIG. 7A was generated. The construct included an inducible iSynPro promoter and IL2 minimal promoter operably linked to a nucleic acid encoding caSTAT5a, a T2A sequence, and a truncated HER2 (Her2t) marker protein; an insulator sequence; a constitutive EF1a promoter operably linked to a nucleic acid encoding an anti-CD19 CAR, a P2A sequence, a selectable DHFRdm protein, a T2A sequence, and a truncated EGFR (EGFRt) marker protein.

Primary human CD8 T cells were electroporated with either (a) a construct encoding an anti-CD19 CAR, (2) the dual promoter construct, or (3) mock. A flow analysis of the transfected cells is shown in FIG. 7B. The transfected cells were co-cultured with Raji/mCherry cells, which express CD19 and an mCherry reporter gene for 24 hours, stained, and underwent flow analysis (FIG. 7B), In an incucyte analysis, transfected cells (effector cells) were co-cultured with Raji/m.Cherry cells (target cells) at an E:T ratio of 1:2. The effector cells were re-challenged with target cells at 60 hours and 126 hours, Levels of expression of the mCherry from the target cells were measured (FIG. 7C), As shown in FIG. 7C, lower levels of normalized mCherry signal was observed with cells cocultured with effector cells containing the dual promoter construct (icaSTATa+CD19CAR) compared to coculture with effector cells containing the anti-CD19 CAR (CD19CAR). This showed that effector cells containing the dual promoter construct (icaSTATa+CD19CAR) were more effective at killing target cells.

### Example 6—Dual Promoter Cassette in a Lentiviral Vector

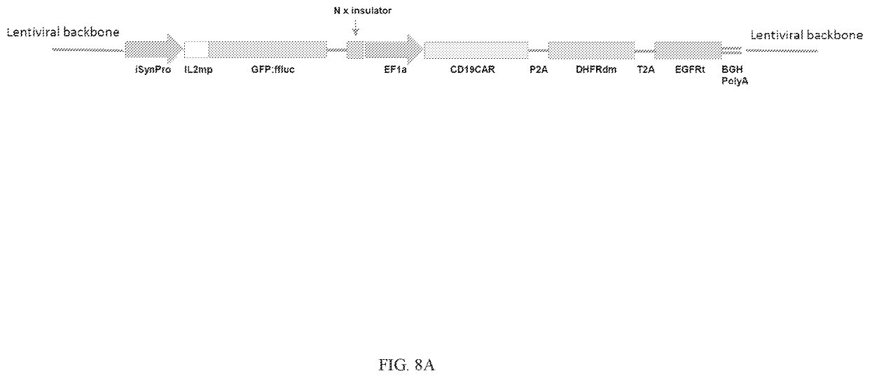

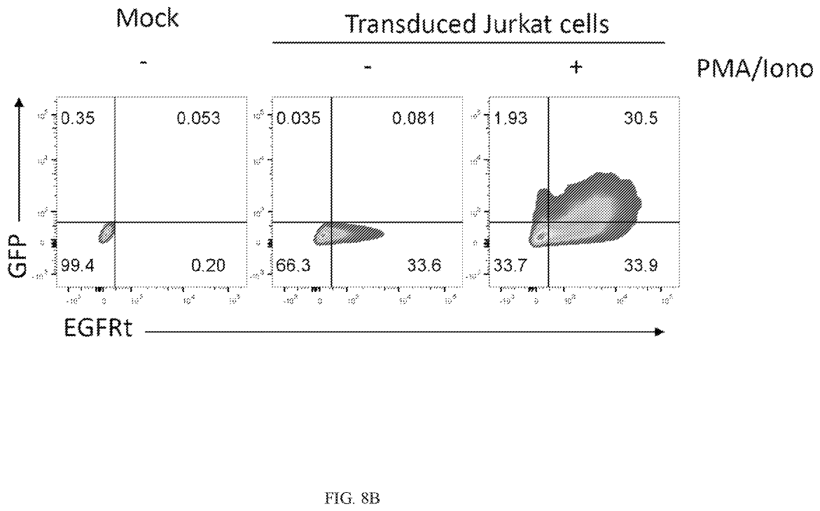

This example relates to construction and use of a dual promoter in a lentiviral vector. A dual promoter construct in a lentiviral vector depicted in FIG. 8A was generated. The construct included an inducible iSynPro promoter and IL2 minimal promoter operably linked to a nucleic acid encoding a GFP reporter gene; an insulator sequence; a constitutive Ma promoter operably linked to a nucleic acid encoding an anti-CD19 CAR, a P2A sequence, a selectable DHFRdm protein, a T2A sequence, and a truncated EGFR (EGFRt) marker protein, and a BGH polyA sequence.

The construct was transfected into Jurkat cells and treated with PMA and ionomycin overnight, stained and analyzed by flow cytometry. Cells treated with PMA and ionomycin expressed GFP indicating that a lentiviral vector containing a dual promoter system was functional and effective.

### Example 7—Logic Gate Systems

A logic gate system similar to the system depicted in FIG. 9 and FIG. 10 are examined. Dual promoter nucleic acids are prepared and introduced into host cells. The nucleic acids include an inducible iSynPro promoter and either a constitutively activated signal transducer and activator of transcription 5a (CA-STAT5a) protein (FIG. 9), a fusion protein of CA-STAT5a and of an estrogen receptor (CA-STAT5aER) (FIG. 10), or an alternative fusion protein of CA-STAT5a and of an estrogen receptor (CA-STAT5bER). Host cells are stimulated with Raji cells (CD19+), or PMA/ionomycin and are analyzed by FACS analysis for GFP and EGFRt expression. Host cells co-cultured with CD19+ cells and treated with tamoxifen express target reporter gene. In contrast, host cells co-cultured with CD19+ cells and not treated with tamoxifen express no target reporter gene.

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1.-97. (canceled)

98. A nucleic acid encoding a dual promoter system, comprising:
a first polynucleotide comprising a first promoter inducible by activation of a receptor, the first promoter operably linked to a polynucleotide encoding a fusion protein comprising a constitutively activated STAT5a and an estrogen receptor, wherein the first promoter comprises a nucleotide sequence having at least 95% identity with the nucleotide sequence set forth in any one of SEQ ID NOs:1-5;
a second polynucleotide comprising a second promoter operably linked to a polynucleotide encoding the receptor; and
wherein a first insulator is located between the first polynucleotide and the second polynucleotide, such that the first insulator is located i) 3′ of the first polynucleotide and 5′ of the second polynucleotide or ii) 5′ of the first polynucleotide and 3′ of the second polynucleotide.

99. The nucleic acid of claim 98, wherein the first promoter comprises the nucleotide sequence set forth in SEQ ID NO:1.

100. The nucleic acid of claim 98, wherein the first promoter further comprises an IL-2 minimal promoter.

101. The nucleic acid of claim 98, wherein the second promoter comprises an EF1α promoter, a PGK promoter, a MND promoter, a MSCV promoter, a CMV promoter, a UBC promoter, a CAGG promoter, or an SV40 promoter.

102. The nucleic acid of claim 98, wherein the first insulator comprises a 3′ HS-A β-globin insulator.

103. The nucleic acid of claim 98, wherein the first insulator comprises a nucleotide sequence having at least 95% identity with the nucleotide sequence set forth in SEQ ID NO:6.

104. The nucleic acid of claim 98, wherein the receptor is activated by the receptor binding a ligand for the receptor.

105. The nucleic acid of claim 98, wherein the receptor comprises a T cell receptor (TCR) or a chimeric antigen receptor (CAR).

106. The nucleic acid of claim 98, further comprising a polynucleotide encoding a selectable marker.

107. The nucleic acid of claim 106, wherein the selectable marker comprises a cell-surface selectable marker or a drug selectable marker.

108. The nucleic acid of claim 98, further comprising a ribosome skip sequence or an internal ribosome entry site (IRES).

109. The nucleic acid of claim 98, wherein a second insulator is located 5′ to either the first polynucleotide or the second polynucleotide and a third insulator is located 3′ to either the first polynucleotide or the second polynucleotide.

110. A cell comprising the nucleic acid of claim 98.

111. The cell of claim 110, wherein the cell is an immune cell or a hematopoietic stem cell.

112. The cell of claim 111, wherein the immune cell is a CD4+ T cell or a CD8+ T cell.

113. A method comprising:
administering the cell of claim 110 to a subject.

114. The method of claim 113, further comprising administering to the subject an inhibitor, an activator, or a molecule that binds the estrogen receptor.

115. The method of claim 114, wherein the inhibitor comprises dasatinib.

116. The method of claim 114, wherein the activator comprises phorbol 12-myristate 13-acetate (PMA) or ionomycin.

117. The method of claim 114, wherein the molecule that binds the estrogen receptor comprises tamoxifen, an analogue of tamoxifen, or a metabolite of tamoxifen.
